(19) United States
(12) Patent Application Publication
(10) Pub. No.: US 2003/0191328 A1
(43) Pub. Date: Oct. 9, 2003

(54) Method for expoxidation of hydrocarbons

(76) Inventors: Ursula Jansen, Neuss (DE); Georg Wiessmeier, Bergisch Gladbach (DE)

Correspondence Address: BAYER POLYMERS LLC, 100 BAYER ROAD, PITTSBURGH, PA, 15205, US

(21) Appl. No.: 10/276,341

(22) PCT Filed: May 7, 2001
(86) PCT No.: PCT/EP01/05136

(30) Foreign Application Priority Data
May 18, 2000 (DE) 100 24 096.8

(51) Int. Cl. C07D 301/06

(52) U.S. Cl. 549/533

(57) ABSTRACT

The invention relates to a method for the epoxidation of hydrocarbons using oxygen. Said method is characterized in that it is carried out in the presence of a mixture containing at least two metals from the group Cu, Ru, Rh, Pd, Os, Ir, Pt, Au, In, Tl, Mn, Ce on a support with a BET surface area of less than 200 m²/g. The invention also relates to the use of a mixture containing at least two metals from the group Cu, Ru, Rh, Pd, Os, Ir, Pt, Au, In, Tl, Mn, Ce on a support with a BET surface area of less than 200 m²/g for the epoxidation of hydrocarbons.

The present invention relates to a process for the epoxidation of hydrocarbons with oxygen, characterised in that the process is performed in the presence of a mixture containing at least two metals from the group Cu, Ru, Rh, Pd, Os, Ir, Pt, Au, In, Tl, Mn, Ce on a support having a BET surface area of less than 200 m²/g and to the use of a mixture containing at least two metals from the group Cu, Ru, Rh, Pd, Os, Ir, Pt, Au, In, Tl, Mn, Ce on a support having a BET surface area of less than 200 m²/g for the epoxidation of hydrocarbons.

Epoxides are an important starting material for the polyurethane industry. There is a range of processes for the production thereof, some of which have also been implemented industrially. Ethylene oxide is produced industrially today by direct oxidation of ethene with air or with gases containing molecular oxygen in the presence of a catalyst containing silver, as described in EP-A-2 933 130. Longer-chain epoxides are generally produced on an industrial scale by using hydrogen peroxide or hypochloride in the liquid phase as oxidising agents. EP-A1-0 930 308 describes, for example, the use of ion-exchanged titanium silicalites as the catalysts with these two oxidising agents.

Another class of oxidation catalysts which permits the oxidation of propene in the gas phase to yield the corresponding epoxide has recently been disclosed by U.S. Pat. No. 5,623,090. In this case, gold on anatase is used as the catalyst, while oxygen, which is used in the presence of hydrogen, acts as the oxidising agent. The system is distinguished by extraordinarily high selectivity (S>95%) with regard to propene oxidation. Disadvantages of the process are low conversion and catalyst deactivation.

Not much is known in the literature about other active components apart from silver and gold for the selective direct oxidation of propene and higher alkenes in the gas phase to yield epoxides.

U.S. Pat. No. 3,644,510 performs the reaction on an Al₂O₃-supported Ir heterogeneous catalyst to yield acetic acid. Depending upon the position of the double bond, higher olefins give rise to ketones or fatty acids (U.S. Pat. No. 3,644,511). In the presence of Rh as supported catalyst, as in U.S. Pat. No. 3,632,833, or of Au, as in U.S. Pat. No. 3,725,482, the principal product is acrolein.

Since none of the catalysts in the public domain had hitherto exhibited satisfactory results with regard to activity and selectivity in the direct oxidation of propene to yield propene oxide, the intention was to investigate other active components as an alternative to known catalysts containing silver and gold. An important condition is that oxidation does not proceed to completion to yield the corresponding acid or the aldehyde or ketone or to yield carbon dioxide.

Mixtures of metals of groups 8-11 of the IUPAC 1986 periodic system are already known in the literature. Cu/Ru mixtures on various supports are used for the hydrogenolysis of alkanes or the hydrogenation of aromatics [Allan J. Hong et al.; J. Phys. Chem., 1987, 91, 2665-2671].

R. S. Drago et al [JACS, 1985, 107, 2898-2901] describe the oxidation of terminal olefins with oxygen to yield the corresponding ketones on unsupported Rh(III)/Cu(II) catalysts in the liquid phase. The formation of epoxides is not disclosed.

T. Inui et al. [J. Chem. Soc., Faraday Trans. 1, 1978, 74, 2490-500] oxidise propene to yield acrolein by means of Cu catalysts, which are modified with Au, Rh, Ag or mixtures thereof. The formation of epoxides is not disclosed.

Supported binary systems of Au and Ru are also already known from the literature (supported on carbon [U.S. Pat. No. 5,447,896 and U.S. Pat. No. 5,629,462], MgO [J. M. Cowley et al, J. Catal.; 1987, 108, 199-207], SiO₂ [Datye et al; Int. Congress Catal. Proc. 8^{th}, 1985 (meeting date 1984), vol. 4, IV587-IV598] or Al₂O₃ [M. Viniegra et al., React. Kinet. Catal. Lett., 1985, 28, 389-94]).

The formation of propene oxide or the use of the catalysts for the direct oxidation of alkenes is also not mentioned for these metal combinations. AuCu systems on SiO₂ were used as long ago as 1976 by Sinfelt et al. [U.S. Pat. No. 3,989,764] for the partial oxidation of propene, isobutene, 1-butene and toluene. Acrolein, methacrolein, methylene acetone and benzene are respectively formed. The formation of propene oxide is not described. Ikeda et al. [Sekiyu Gakkaishi; 1967, 10, 119-23, from HCA 68:113989, abstract] have made a similar report, in which acrolein is produced in the gas phase from propene. The CuAu catalyst is applied onto porcelain.

It has now surprisingly been found that propene oxide may be produced by direct oxidation of propene with oxygen or air with mixtures of various metals. This is all the more unusual as, according to the literature, oxidation does not stop at the epoxide stage, but instead the corresponding acids, ketones or aldehydes are formed.

The present invention provides a process for the epoxidation of hydrocarbons with oxygen, characterised in that the process is performed in the presence of a mixture containing at least two metals from the group Cu, Ru, Rh, Pd, Os, Ir, Pt, Au, In, Tl, Mn, Ce on an inert support having a BET surface area of less than 200 m²/g.

The term hydrocarbon is taken to mean unsaturated or saturated hydrocarbons such as olefins or alkanes, which may also contain heteroatoms such as N, O, P, S or halogens. The organic component to be oxidised may be acyclic, monocyclic, bicyclic or polycyclic and may be monoolefinic, diolefinic or polyolefinic. In organic components having two or more double bonds, the double bonds may be present in conjugated and non-conjugated form. The hydrocarbons oxidised are preferably those from which oxidation products are formed which have a partial pressure at the reaction temperature which is sufficiently low to allow continuous removal of the product from the catalyst.

Unsaturated and saturated hydrocarbons having 2 to 20, preferably 3 to 10 carbon atoms, are preferred, in particular propene, propane, isobutane, isobutylene, 1-butene, 2-butene, cis-2-butene, trans-2-butene, 1,3-butadiene, pentene, pentane, 1-hexene, 1-hexane, hexadiene, cyclohexene, benzene.

The oxygen may be used in the most varied forms, such as molecular oxygen, air and nitrogen oxide. Molecular oxygen is preferred. Suitable mixtures are preferably binary or ternary mixtures of the metals Cu, Ru, Rh, Pd, Os, Ir, Pt, Au, In, TI, Ce, wherein the contents of the individual metals are in each case within the range from 0-100 rel. wt. % and, unremarkably, add up to 100%.

The following mixtures are preferred, CuRu, TlMn, CuRh, IrRu, AuRu, MnCu, RuIr as well as CuRuPd, CuRuIn, CuRuTl, CuRuMn, CuRuAu, CuRuIr, CuRuCe, MnCuIn, MnCuAu, MnCuCe, MnTlCu, MnTlAu, MnTlIn, MnTlPd, MnTlRh, MnTlPt.

The supports comprise compounds from the class Al₂O₃, SiO₂, CeO₂, TiO₂ having BET surface areas of <200 m²/g, preferably of <100 m²/g, particularly preferably of 10 m²/g and very particularly preferably of <1 m²/g.

Porosity is advantageously 20-60%, in particular 30-50%.

The particle size of the supports is determined by the process conditions of the gas phase oxidation and is conventionally in the range from 1/10th to 1/20th of the reactor diameter.

Specific surface area is determined in the conventional manner according to Brunauer, Emmett and Teller, J. Am. Chem. Soc. 1938, 60, 309; porosity by mercury porosimetry and the particle size of the metal particles on the surface of the support by electron microscopy.

The concentration of metal on the support should generally be in the range from 0.001 to 50 wt. %, preferably from 0.001 to 20 wt. %, very particularly preferably from 0.01 to 5 wt. %.

Production of the metal particles on the support is not restricted to a single method. Several examples of processes may be mentioned in this connection for the production of metal particles, such as deposition-precipitation, as described in EP-B-0 709 360 on page 3, lines 38 et seq., impregnation in solution, incipient wetness process, colloid process, sputtering, CVD, PVD.

The incipient wetness process is taken to mean the addition of a solution containing soluble metal compounds to the support material, wherein the volume of the solution on the support is less than or equal to the pore volume of the support. The support thus remains macroscopically dry. Solvents which may be used for the incipient wetness process comprise any solvents in which the metal precursors are soluble, such as water, alcohols, (crown) ethers, esters, ketones, halogenated hydrocarbons, etc.

The support is preferably impregnated with a solution containing the metal ions and then dried, calcined and reduced. The solution may furthermore additionally contain components known to the person skilled in the art which may increase the solubility of the metal salt or salts in the solvent and/or modify the redox potential of the metals and/or modify the pH value. Components which may in particular be mentioned are ammonia, amines, diamines, hydroxyamines and acids, such as HCl, HNO₃, H₂SO₄, H₃PO₄.

1. Impregnation may, for example, be performed by the incipient wetness method, but is not restricted thereto. The incipient wetness process may here comprise the following steps:

single surface-modification with a metal and/or repeated surface-modification with another metal,

single surface-modification with a proportion of the metals or with all the metals in a single step,

repeated surface-modification with two or more metals in one or more successive steps,

repeated surface-modification with two or more metals alternately in one or more steps.

2. Drying of the support with the active components obtained according to 1 at a temperature of approximately 40 to approximately 200° C. at standard pressure or also reduced pressure. At standard pressure, drying may be performed under an atmosphere of air or also under an inert gas atmosphere (for example Ar, N₂, He et al.). Drying time is in the range from 2-24 h, preferably from 4-8 h.

3. Calcination of the catalyst precursors obtained according to 2 under an inert gas atmosphere and subsequently or exclusively under a gas atmosphere containing oxygen. The oxygen contents in the gas stream advantageously range from 0 to 21 vol. %, preferably from 5-15 vol. %. The calcination temperature is adapted to the metal mixture and is accordingly generally in the range from 400 to 600° C., preferably at 450-550° C., particularly preferably at 500° C.

4. Reduction of the catalyst precursors obtained according to 2 and/or 3 at elevated temperatures under a nitrogen atmosphere containing hydrogen. The content of hydrogen may be between 0-100 vol. %, but preferably at 0-25, particularly preferably at 5 vol. %. Reduction temperatures are adapted to the particular metal mixture and are between 100 and 600° C.

It may be advantageous to admix conventional promoters or moderators, such as alkaline earth and/or alkali metal ions as hydroxides, carbonates, nitrates, chlorides of one or more alkaline earth and/or alkali metals, to the metal mixture. These substances are described in EP-A1-0 933 130 on page 4, lines 39 et seq., which is simultaneously included in the present application as a reference to US practice.

The epoxidation process is conventionally performed under the following conditions, preferably in the gas phase:

The molar quantity of the hydrocarbon used relative to the total number of moles of hydrocarbon, oxygen and optionally diluent gas and the relative molar ratio of the components may be varied within broad ranges and is generally determined by the explosion limits of the hydrocarbon/oxygen mixture. The process is generally performed above or below the explosion limit.

The hydrocarbon content, relative to the total moles of hydrocarbon and oxygen, is typically <2 mol % or >78 mol %. In the case of modes of operation below the explosion limit, hydrocarbon contents in the range from 0.5-2 mol % are preferably selected, while in the case of modes of operation above the explosion limit, contents of 78-99 mol % are preferably selected. The ranges of 1-2 mol % and of 78-90 mol % are particularly preferred in each case. Hydrocarbon is preferably used in an excess relative to the oxygen used (on a molar basis).

The molar content of oxygen, relative to the total number of moles of hydrocarbon, oxygen and diluent gas, may be varied within broad limits. The oxygen is preferably used in a molar deficit relative to the hydrocarbon. Oxygen is preferably used in the range of 1-21 mol %, particularly preferably of 5-21 mol %, relative to the total moles of hydrocarbon and oxygen.

In addition to hydrocarbon and oxygen, a diluent gas may optionally also be used, such as nitrogen, helium, argon, methane, carbon dioxide, carbon monoxide or similar gases which exhibit largely inert behaviour. Mixtures of the described inert components may also be used. Addition of the inert components is favourable for dissipating the heat liberated during this exothermic oxidation reaction and from a safety standpoint. In this case, the above described composition of the starting gas mixtures is also possible within the explosion range, i.e. the relative ratio of hydrocarbon and oxygen may be between 0.5:99.5 and 99.5:0.5 mol %.

The contact time between hydrocarbon and catalyst is generally in the range from 5-60 seconds.

The process is generally performed at temperatures in the range from 120-300° C., preferably of 180-250° C.

### EXAMPLES

### Example 1

One possible option for producing an active catalyst for PO production comprises, for example, dissolving 77.6 mg of copper nitrate and 3.59 g of an approximately 14% ruthenium nitrosyl nitrate solution in 2 ml of water, adding the solution to approximately 10 g of Al₂O₃ and allowing the solution to be absorbed. The resultant solid is dried overnight at 100° C. in a vacuum drying cabinet at a vacuum of approximately 15 mm Hg.

The resultant precursor is finally reduced for 12 h at 500° C. with 10 vol. % of H₂ in N₂ at 60 l/h.

After reduction, 10 g of the resultant catalyst are investigated in a continuously operated fixed bed reactor with an educt gas composition of 79 vol. % propene and 21 vol. % oxygen at a residence time of approximately 20 sec. At an internal temperature of 217° C., PO contents of 680 ppm are determined in the exit gas stream.

### Example 2

One possible option for producing an active catalyst for PO production comprises, for example, dissolving 77.6 mg of copper nitrate in 5-6 ml of water, adding the solution to approximately 10 g of Al₂O₃ and allowing the solution to be absorbed. The resultant solid is dried for 12 h at 60° C. in a vacuum drying cabinet at a vacuum of approximately 15 mm Hg. The solid is then surface-modified in the same manner 6 times with a ruthenium nitrosyl nitrate solution containing approximately 1.5 wt. % Ru in accordance with the absorption capacity of the support. Drying is performed as above for 4 hours between each surface-modification.

The resultant precursor is finally reduced for 12 h at 500° C. with 10 vol. % of H₂ in N₂ at 60 l/h.

After reduction, 10 g of the resultant catalyst are investigated in a continuously operated fixed bed reactor with an educt gas composition of 79 vol. % propene and 21 vol. % oxygen at a residence time of approximately 20 sec. At an internal temperature of 200° C., PO contents of 300 ppm are determined in the exit gas stream.

### Example 3

One possible option for producing an active catalyst for PO production comprises, for example, dissolving 77.6 mg of copper nitrate in 5-6 ml of water, adding the solution to approximately 10 g of Al₂O₃ and allowing the solution to be absorbed. The resultant solid is dried for 12 h at 60° C. in a vacuum drying cabinet at a vacuum of approximately 15 mm Hg. The solid is then surface-modified in the same manner with 2.5 g of a ruthenium nitrosyl nitrate solution containing approximately 20 wt. % Ru and drying is then performed as described in Example 1. The resultant precursor is finally reduced for 12 h at 500° C. with 10 vol. % of H₂ in N₂ at 60 l/h.

After reduction, 10 g of the resultant catalyst are investigated in a continuously operated fixed bed reactor with an educt gas composition of 79 vol. % propene and 21 vol. % oxygen at a residence time of approximately 20 sec. At an internal temperature of 200° C., PO contents of 280 ppm are determined in the exit gas stream.

### Example 4

Another possible option for producing an active catalyst for PO production comprises, for example, adding 7.4 g of a 10% rhodium nitrate solution to approximately 10 g of Al₂O₃ and allowing the solution to be absorbed. The resultant solid is dried for 4 h at 100° C. in a vacuum drying cabinet at a vacuum of approximately 15 mm Hg. The solid is then surface-modified in the same manner with 1.3 g of a ruthenium nitrosyl nitrate solution containing approximately 20 wt. % Ru and drying is then performed as described in a vacuum drying cabinet for 12 h. The resultant precursor is finally reduced for 4 h at 500° C. with 10 vol. % of H₂ in N₂ at 60 l/h.

After reduction, 1 g of the resultant catalyst is investigated in a continuously operated fixed bed reactor with an educt gas composition of 79 vol. % propene and 21 vol. % oxygen at a residence time of approximately 20 sec. At an internal temperature of approximately 199° C., PO contents of 360 ppm are determined in the exit gas stream.

### Example 5

An alternative option for producing an active catalyst for PO production comprises, for example, dissolving 343 mg of thallium nitrate in 5 g of water and impregnating approximately 10 g of Al₂O₃ with the resultant solution. The solid is allowed to absorb the solution while being kept in constant motion and the resultant solid is dried for 4 h at 100° C. in a vacuum drying cabinet at a vacuum of approximately 15 mm Hg. The solid is then surface-modified in the same manner with a solution produced from 776 mg of copper(II) nitrate and 5 g of water and then dried overnight at 100° C. in a vacuum drying cabinet at approximately 15 nun Hg.

The resultant precursor is finally reduced for 12 h at 500° C. with 10 vol. % of H₂ in N₂ at 60 l/h.

After reduction, 1 g of the resultant catalyst is investigated in a continuously operated fixed bed reactor with an educt gas composition of 79 vol. % propene and 21 vol. % oxygen at a residence time of approximately 20 sec. At an internal temperature of 228° C., PO contents of 380 ppm are measured in the exit gas stream.

### Example 6

2.5 g of a 20% ruthenium nitrosyl nitrate solution are dissolved in 3 g of water and approximately 10 g of Al₂O₃ are impregnated with the resultant solution. The solid is allowed to absorb the solution while being kept in constant motion and the resultant solid is dried for 4 h at 100° C. in a vacuum drying cabinet at a vacuum of approximately 15 mm Hg. The solid is then surface-modified in the same manner with a solution produced from 109 mg of 24% hexachloroiridic acid solution and 4.5 g of water and then dried overnight at 100° C. in a vacuum drying cabinet at approximately 15 mm Hg.

The resultant precursor is finally reduced for 12 h at 500° C. with 10 vol. % of H₂ in N₂ at 60 l/h.

After reduction, 1 g of the resultant catalyst is investigated in a continuously operated fixed bed reactor with an educt gas composition of 79 vol. % propene and 21 vol. % oxygen at a residence time of approximately 20 sec. At an internal temperature of 208° C., PO contents of 540 ppm are measured in the exit gas stream.

### Example 7

343 mg of thallium nitrate are dissolved in 5 g of water and 10 g of Al₂O₃ are impregnated with the resultant solution. The solid is allowed to absorb the solution while being kept in constant motion and the resultant solid is dried for 4 h at 100° C. in a vacuum drying cabinet at a vacuum of approximately 15 mm Hg. The solid is then surface-modified in the same manner with a solution produced from 1.3 g of a 20% ruthenium nitrosyl nitrate solution and then dried overnight at 100° C. in a vacuum drying cabinet at approximately 15 mm Hg.

The resultant precursor is finally reduced for 12 h at 500° C. with 10 vol. % of H₂ in N₂ at 60 l/h.

After reduction, 1 g of the resultant catalyst is investigated in a continuously operated fixed bed reactor with an educt gas composition of 79 vol. % propene and 21 vol. % oxygen at a residence time of approximately 20 sec. At an internal temperature of 211° C., PO contents of 390 ppm are measured in the exit gas stream.

### Example 8

17.86 g of copper nitrate are dissolved in 103 g of water and 230 g of Al₂O₃ are impregnated with the resultant solution. The solid is allowed to absorb the solution while being kept in constant motion and the resultant solid is dried for 4 h at 100° C. in a vacuum drying cabinet at a vacuum of approximately 15 mm Hg. The solid is then surface-modified in the same manner with a solution produced from 43.52 g of a 14% ruthenium nitrosyl nitrate solution and 71 g of water and then dried overnight at 100° C. in a vacuum drying cabinet at approximately 15 mm Hg.

The resultant precursor is reduced for 4 h at 500° C. with 10 vol. % of H₂ in N₂ at 60 l/h.

5 g of the resultant solid are then surface-modified with a solution prepared from 6 mg of palladium nitrate in 2.25 g of water and dried overnight at 100° C. in a vacuum drying cabinet.

The resultant precursor is finally reduced for 8 h at 500° C. with 10 vol. % of H₂ in N₂ at 60 l/h.

After reduction, 1 g of the resultant catalyst is investigated in a continuously operated fixed bed reactor with an educt gas composition of 79 vol. % propene and 21 vol. % oxygen at a residence time of approximately 20 sec. At an internal temperature of 220° C., PO contents of 745 ppm are measured in the exit gas stream.

### Example 9

27.6 g of manganese nitrate are dissolved in 103.5 g of water and 230 g of Al₂O₃ are impregnated with the resultant solution. The solid is allowed to absorb the solution while being kept in constant motion and the resultant solid is dried for 4 h at 100° C. in a vacuum drying cabinet at a vacuum of approximately 15 mm Hg. The solid is then surface-modified in the same manner with a solution produced from 7.9 g of thallium nitrate and 103.5 g of water and then dried overnight at 100° C. in a vacuum drying cabinet at approximately 15 mm Hg. The resultant precursor is reduced for 4 h at 500° C. with 10 vol. % of H₂ in N₂ at 60 l/h.

5 g of the resultant solid are then surface-modified with a solution prepared from 259 mg of copper nitrate in 2.25 g of water and dried overnight at 100° C. in a vacuum drying cabinet.

The resultant precursor is finally reduced for 8 h at 500° C. with 10 vol. % of H₂ in N₂ at 60 l/h.

After reduction, 1 g of the resultant catalyst is investigated in a continuously operated fixed bed reactor with an educt gas composition of 79 vol. % propene and 21 vol. % oxygen at a residence time of approximately 20 sec. At an internal temperature of 240° C., PO contents of 1984 ppm are measured in the exit gas stream.

### Example 10

2.76 g of manganese nitrate are dissolved in 103.5 g of water and 230 g of Al₂O₃ are impregnated with the resultant solution. The solid is allowed to absorb the solution while being kept in constant motion and the resultant solid is dried for 4 h at 100° C. in a vacuum drying cabinet at a vacuum of approximately 15 mm Hg. The solid is then surface-modified in the same manner with a solution produced from 33.92 g of copper nitrate and 95 g of water and then dried overnight at 100° C. in a vacuum drying cabinet at approximately 15 mm Hg.

The resultant precursor is reduced for 8 h at 500° C. with 10 vol. % of H₂ in N₂ at 60 l/h.

5 g of the resultant solid are then surface-modified with a solution prepared from 6 mg of a 43.5% tetrachlorogold solution in 2.25 g of water and dried overnight at 100° C. in a vacuum drying cabinet.

The resultant precursor is finally reduced for 8 h at 500° C. with 10 vol. % of H₂ in N₂ at 60 l/h.

After reduction, 1 g of the resultant catalyst is investigated in a continuously operated fixed bed reactor with an educt gas composition of 79 vol. % propene and 21 vol. % oxygen at a residence time of approximately 20 sec. At an internal temperature of 230° C., PO contents of 982 ppm are measured in the exit gas stream.

1. A process for the epoxidation of hydrocarbons with oxygen, characterised in that the process is performed in the presence of a mixture containing at least two metals from the group Cu, Ru, Rh, Pd, Os, Ir, Pt, Au, In, TI, Mn, Ce on a support having a BET surface area of less than 200 m²/g.

2. A process according to claim 1, characterised in that the BET surface area is less than 100 m²/g.

3. A process according to claim 1 or 2, characterised in that the support is Al₂O₃.

4. A process according to any one of claims 1 to 3, characterised in that the hydrocarbon is selected from the group propene and butene.

5. A process according to any one of claims 1 to 4, characterised in that one or more of the metal mixtures CuRu, TlMn, CuRh, IrRu, AuRu, MnCu, Rulr as well as CuRuPd, CuRuIn, CuRuTl, CuRuMn, CuRuAu, CuRuIr, CuRuCe, MnCuIn, MnCuu, MnCuCe, MnTlCu, MnTlAu, MnTlIn, MnTlPd, MnTlRh, MnTIPt is/are used.

6. Use of a mixture containing at least two metals from the group Cu, Ru, Rh, Pd, Os, Ir, Pt, Au, In, TI, Mn, Ce on a support having a BET surface area of less than 200 m²/g as a catalyst for the epoxidation of hydrocarbons.

7. Use according to claim 6, characterised in that the metal mixture is selected from the group CuRu, TlMn, CuRh, IrRu, AuRu, MnCu, RuIr as well as CuRuPd, CuRuin, CuRuTl, CuRuMn, CuRuAu, CuRuIr, CuRuCe, MnCuIn, MnCuAu, MnCuCe, MnTlCu, MnTlAu, MnTlIn, MnTlPd, MnTlRh, MnTlPt.
